(19) United States
(12) Patent Application Publication
(10) Pub. No.: US 2004/0059429 A1
(43) Pub. Date: Mar. 25, 2004

(54) Mechanically attached elastomeric cover for prosthesis

(76) Inventors: Uri Amin, Kiryat Tivon (IL); Yossi Tuval, Netanya (IL); Raphael Benary, Tel Aviv (IL); Michael Earl Kaufman, Sandy, UT (US)

Correspondence Address: Dekel Patent Ltd., Beit HaRofim, Room 27, 18 Menuha VeNahala Street, Rehovot, IL

(21) Appl. No.: 10/663,768

(22) Filed: Sep. 17, 2003

Related U.S. Application Data
(60) Provisional application No. 60/411,932, filed on Sep. 20, 2002.
(60) Provisional application No. 60/426,345, filed on Nov. 15, 2002.
(60) Provisional application No. 60/462,327, filed on Apr. 14, 2003.

(51) Int. Cl. A61F 2/28; A61F 2/32

(52) U.S. Cl. 623/23.51; 623/22.11

(57) ABSTRACT

A prosthetic orthopedic implant assembly including an implant body, and an elastomeric cover connected to the implant body by a mechanical fastener.

### CROSS-REFERENCE TO RELATED APPLICATIONS

This application claims priority under 35 USC §119 to U.S. Provisional Patent Application Serial No. 60/411,932, filed on Sep. 20, 2002, U.S. Provisional Patent Application Serial No. 60/426,345, filed on Nov. 15, 2002, and U.S. Provisional Patent Application Serial No. 60/462,327, filed on Apr. 14, 2003, all of which are incorporated herein by reference.

### FIELD OF THE INVENTION

The present invention relates generally to implantable prostheses including an elastomeric contact surface and methods of making same, and particularly to prostheses having an elastomeric cover mechanically attached thereto.

### BACKGROUND

Implantable prostheses can be used in surgical procedures to replace joints or parts of joints. For example, an artificial ball can be used to replace the humeral head of a shoulder joint in arthroplasty surgery. The prosthesis may include a soft-cushioned ball (or “head”), typically constructed of a metal ball covered by an elastomeric shell, which replaces the naturally articulating surface of the shoulder joint.

Failure of soft-cushioned heads may arise from dislodgement of the elastomeric shell from the underlying hard ball or from tearing of the elastomeric shell, which can cause total failure of the prosthesis.

An elastomeric shell is typically bonded to a hard ball (which may in some cases be porous), by means of glue, cement or in situ hardening of the elastomer. Available glues and cements have typically failed after a time shorter than the expected useful life of the elastomeric shell or the hard ball, causing dislodgement and shortening the useful life of the implant. In situ hardening on the hard ball has also led to the same result.

The bonded attachment of the elastomeric shell to the hard ball may also lead to tearing of the elastomeric shell while it is still adhered to the hard ball. The tearing may result from the bonded attachment that leaves no possibility for translational motion of any portion of the elastomeric shell with respect to the surface of the hard ball. As a result, uneven pressure on the elastomeric shell can cause tearing and needlessly shorten the useful life of the implant.

### SUMMARY

In a first general aspect, a prosthetic orthopedic implant assembly includes an implant body and an elastomeric cover connected to said implant body by a mechanical fastener.

Implementations may include one or more of the following features. The mechanical fastener may include a tongue-and-groove connection between the implant body and said elastomeric cover. The tongue-and-groove connection may include at least one indentation formed on an exterior surface of the implant body and at least one protrusion formed on an interior surface of the elastomeric cover, where the at least one protrusion is received in said at least one indentation. The at least one indentation includes at least a partial annular groove formed on the exterior surface of the implant body.

Further advantages are apparent from the description and the drawings. The implementations mentioned herein can be used either individually or collectively in arbitrary combination. The implementations are not to be understood as an exhaustive enumeration, but rather have exemplary character for describing the invention.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the present invention only

In the drawings:

FIG. 1 is a cross-sectional view of a prosthetic orthopedic implant assembly;

FIG. 1A is a cross sectional view of a portion of the implant of FIG. 1, showing a fluid sealing structure of the implant assembly;

FIG. 2A is a bottom view of another prosthetic orthopedic implant assembly;

FIG. 2B is a cross-sectional view of the prosthetic orthopedic implant assembly of FIG. 2A, taken along lines A-A in FIG. 2A;

FIG. 3 is a cross-sectional view of yet another prosthetic orthopedic implant assembly; and

FIG. 4 is a cross-sectional view of still another prosthetic orthopedic implant assembly.

### DESCRIPTION OF EMBODIMENTS

As is described more in detail hereinbelow, an elastomeric cover is placed onto an implant body and fixed in place by a mechanical fastener.

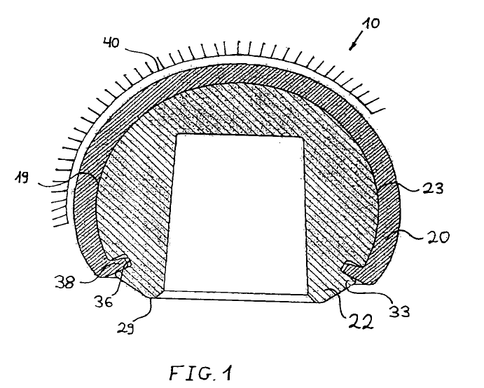

Referring now to FIG. 1, a prosthetic orthopedic implant assembly 10 includes an implant body 22, which may have any degree of rigidity, having an exterior surface 23, and an elastomeric cover 20 connected to the implant body 22 by a mechanical fastener. Elastomeric cover 20 may serve to reduce contact stress between implant body 22 and a complementary articulating surface 40. Implant body 22 can be shaped like a ball, and may be useful in a ball and socket joint. A “ball” refers to at least a portion of a three-dimensional object having a convexly curved contour, such as, but not limited to, a sphere, capable of articulation within a “socket” (i.e., a portion of a three-dimensional shape having a concavely curved contour). The ball may have a radius r, whose size may vary depending on where the ball is used and/or according to the patient's size. For example, the radius of the ball used in an adult patient shoulder may be greater than that used in a hip of the same adult patient. Children may require smaller values of r for a given joint than adults. Further, the portion of the sphere used in the ball also varies depending on where it is to be used. For example 220-280 degrees of a full sphere may be used in a hip while 20-90 degrees of a full sphere may be used in a shoulder.

The material hardness of the elastomeric cover 20 may be between about 60 Shore A to about 65 Shore A. More particularly, the material hardness may be greater than about 60 Shore A, greater than about 65 Shore A, greater than about 70 Shore A, greater than about 75 Shore A, greater than about 80 Shore A, greater than about 85 Shore A, greater than about 90 Shore A, or greater than about 95 Shore A. The material hardness may also be less than about 105 Shore A, less than about 95 Shore A, less than about 90 Shore A, less than about 85 Shore, less than about 80 Short A, less than about 75 Shore A, less than about 70 Shore A, or less than about 65 Shore A. The elastomeric cover 20 may have an elastic modulus of between about 10 to about 150 MPa. More particularly, the material may have an elastic modulus of greater than about 10 MPa, greater than about 30 MPa, greater than about 50 MPa, greater than about 70 MPa, greater than about 90 MPa, greater than about 110 MPa, greater than about 130 MPa, and may have an elastic modulus of less than about 150 MPa, less than about 130 MPa, less than about 110 MPa, less than about 90 MPa, less than about 70 MPa, less than about 50 MPa, or less than about 30 MPa. The outside surface of the elastomeric cover 20 may have a smooth surface or it may have a non-smooth surface, such as some modifications to a generally smooth surface to improve its lubrication quality when in use, e.g., grooves or a slightly rough texture. Scoring the surface slightly with shallow grooves may facilitate lubrication by providing passageways for retaining synovial fluid on the surface.

The implant body 22 can include a lower end 29 at least partially surrounded by a ridge 33, which may help prevent elastomeric cover 20 from becoming detached from exterior surface 23 during articulation within complementary articulating surface 40.

Still referring to FIG. 1, the mechanical fastener that fastens elastomeric cover 20 to implant body 22 is a tongue-and-groove connection between implant body 22 and elastomeric cover 20. The tongue-and-groove connection may include at least one indentation 36 on exterior surface 23 and at least one protrusion 38 on an interior surface 19 of elastomeric cover 20. The one or more protrusions 38 are designed, constructed and arranged to be received in the one or more indentations 36 in a sufficiently snug manner so as to mechanically secure cover 20 to curved surface 23 of implant body 22 during articulation within complementary articulating surface 40. The at least one indentation 36 may be formed as an annular groove about the exterior surface 23, or a partial annular groove. Alternately or additionally, the one or more indentations 36 may be on an interior surface 19 of elastomeric cover 20, and the one or more protrusions 38 may be on curved surface 23 of implant body 22.

Referring now to FIG. 1A, a fluid sealing structure 44 of the implant assembly 10 includes a tooth (e.g., at least partial annular in shape) protruding from indentation 36 that sealingly presses against protrusion 38. Fluid sealing structure 44 prevents ingress of fluid between elastomeric cover 20 and implant body 22.

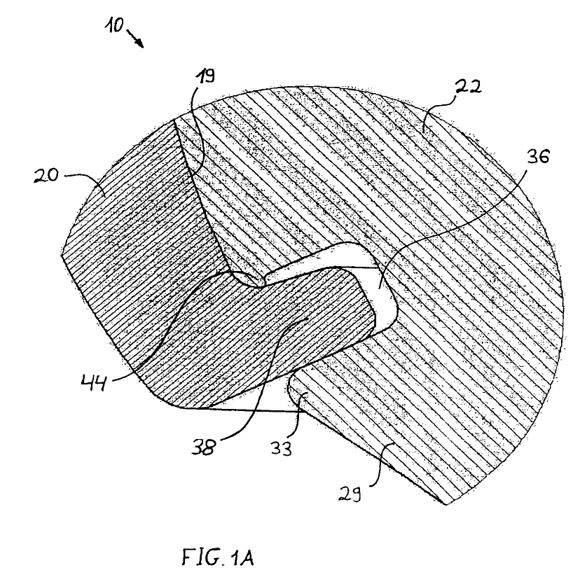

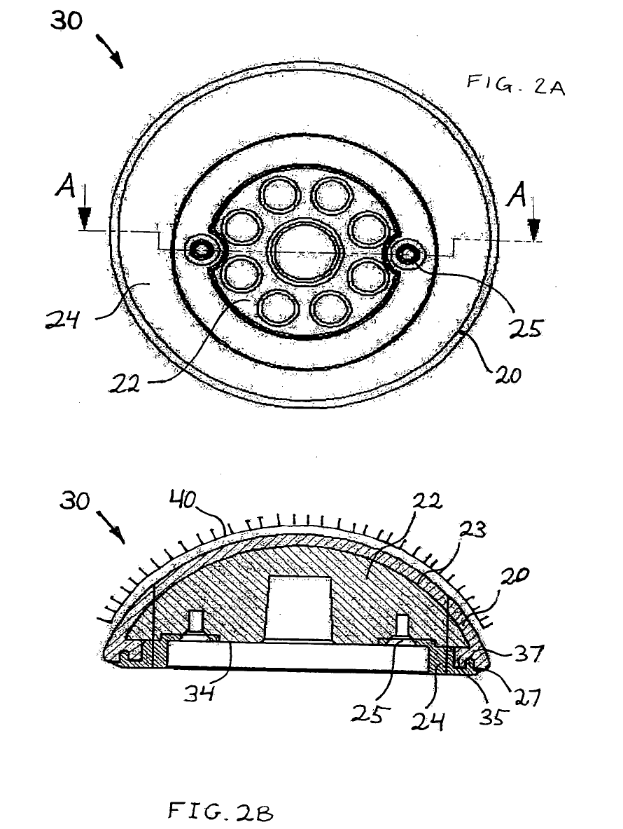

Referring now to FIGS. 2A and 2B, a prosthetic orthopedic implant assembly 30 includes an elastomeric cover 20 that can include a lip 35 that faces radially inwards and which may be formed with an at least partial annular groove 37. Elastomeric cover 20 fits over the exterior surface 23 of implant body 22 and may be fastened thereto with a mechanical fastener, such as but not limited to, a retaining element, e.g., a retaining ring 24. (The retaining element may have other shapes than a ring shape, such as but not limited to, rectangular. The terms retaining element and retaining ring will be used interchangeably, it being understood that ring does not limit the shape of the retaining element.) Retaining ring 24 may be secured to a non-articulating (bottom) surface 34 by means of one or more fasteners 25, such as but not limited to, screws, rivets, bolts and the like. Additionally or alternatively, retaining ring 24 may be secured to non-articulating (bottom) surface 34 by welding, bonding or other joining methods. Non-articulating (bottom) surface 34 may be a flat surface. Retaining ring 24 may be formed with an at least partial annular ridge 27 that mates with the at least partially annular groove 37. (Alternatively, groove 37 may be formed in implant body 22.)

During articulation of the joint, elastomeric cover 20 articulates (moves) with respect to complementary articulating surface 40. The attachment of retaining ring 24 to lip 35 and implant body 22 may prevent elastomeric cover 20 from becoming detached from surface 23 of implant body 22 during articulation within complementary articulating surface 40.

Retaining ring 24 may be constructed of any material, such as the same or different material of which implant body 22 is constructed. Typically implant body 22 is constructed of a ceramic or metallic material, although other materials may be employed as well. Retaining ring 24 may be constructed of a material with the same, less or greater hardness (or other properties) than implant body 22 or elastomeric cover 20.

Retaining ring 24 may permit a slight amount of local relative translational or rotational motion between elastomeric cover 20 and implant body 22, such as a result of friction with surrounding tissue (e.g., the socket wall) or when pressure is applied to a particular point on the elastomeric cover 20. This slight local relative movement can reduce the possibility of tearing the elastomeric cover 20.

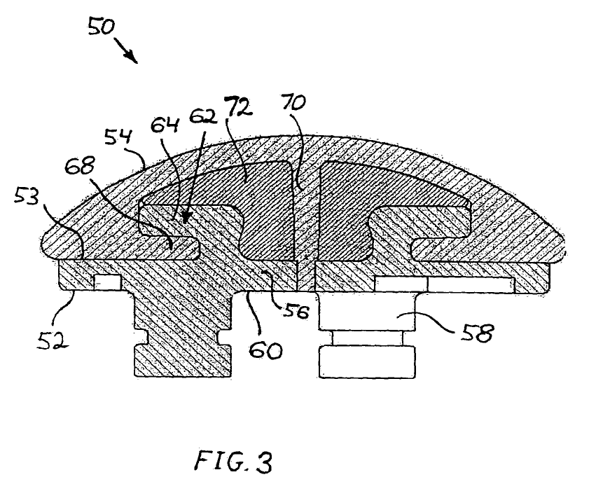

Referring now to FIG. 3, implant assembly 50 may include an implant body 52, which may have any degree of rigidity, having an exterior (top, in the sense of the drawing) surface 53, and an elastomeric cover 54 mechanically connectable to the implant body 52. Implant body 52 may include a plate portion 56 having one or more mounting members 58 protruding from an interior (bottom) surface 60 thereof. Mounting members 58 may be in the form of round pegs for installation in other prosthetic elements or in bone structure, for example. Mounting members 58 may alternatively or additionally include threaded fasteners, threaded inserts, spikes and the like.

The exterior surface 53 of plate portion 56 may include one or more fasteners 62 for fastening elastomeric cover 54 to implant body 52. Fastener 62 may include an at least partial annular lip 64 generally parallel (although not necessarily parallel) to plate portion 56 and spaced therefrom by an at least partial annular spacer ring 66. Elastomeric cover 54 may include an inwardly facing lip 68 that snugly fits into the gap between lip 64 and plate portion 56, thereby mechanically fastening elastomeric cover 54 to implant body 52.

The at least partially annular lip 64, plate portion 56 and the at least partial spacer ring 66 may be formed of one integral piece. Optionally, the at least partial annular lip 64, plate portion 56 and the at least partial spacer ring 66 may be formed of separate pieces, and the elastomeric cover 54 may include a partition wall 70 that snugly fits into the gap between the individual plate portions 56.

Optionally, implant assembly 50 may include an inner core 72 inwards of elastomeric cover 54, which is held in place by the mechanical attachment of elastomeric cover 54 to implant body 52. Inner core 72 may be constructed of a material with different or same properties as elastomeric cover 54. For example, inner core 72 may be made of a material that is more compliant and resilient than elastomeric cover 54, which may impart increased compliancy to implant assembly 50.

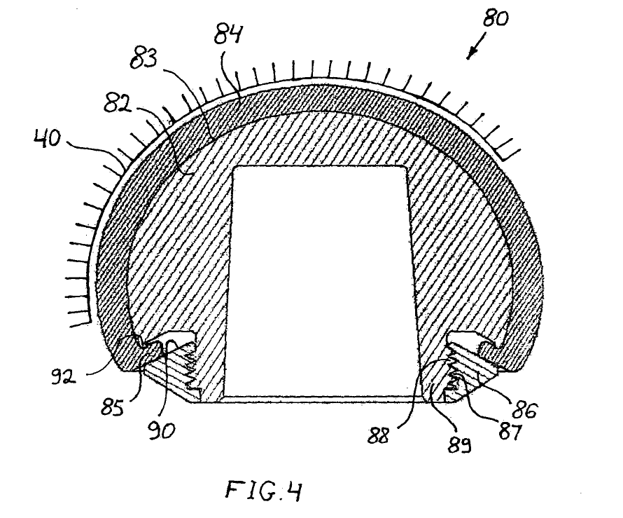

Referring now to FIG. 4, implant assembly 80 may include an implant body 82, which may have any degree of rigidity, having an exterior (top, in the sense of the drawing) surface 83, and an elastomeric cover 84 mechanically connectable to the implant body 82. Implant body 82 is generally shaped like a ball, and may be useful in a ball and socket joint.

The mechanical fastener may be a threaded fastener (also referred to as a threaded connection) that fastens elastomeric cover 84 to implant body 82. The threaded connection may include a threaded ring 86 that mates with complementary formed threads 88 on a stem 89 of implant body 82. The threaded ring 86 may include internal threads 87 whereas the complementary formed threads 88 are external threads. Alternatively, the threaded ring 86 may include external threads whereas the complementary formed threads 88 may be internal threads.

As similarly shown in FIG. 1, the elastomeric cover 84 may have one or more protrusions 85, which may be formed as an at least partial ring about elastomeric cover 84. The threaded ring 86 may be tightened against protrusion 85 to secure elastomeric cover 84 against implant body 82. The threaded ring 86 may have a chamfer 90 that abuts against protrusion 85. As similarly shown in FIG. 1A, fluid sealing structure 92 may be provided, such as a tooth (e.g., at least partial annular in shape) protruding from implant body 82 that sealingly presses against protrusion 85. Fluid sealing structure 92 may help prevent ingress of fluid between elastomeric cover 84 and implant body 82.

### Other Embodiments

A number of embodiments of the invention have been described. Nevertheless, it will be understood that various modifications may be made. Accordingly, other embodiments are within the scope of the following claims.

What is claimed:

1. A prosthetic orthopedic implant assembly comprising:
an implant body; and
an elastomeric cover connected to said implant body by a mechanical fastener.

2. The prosthetic orthopedic implant assembly according to claim 1, wherein the mechanical fastener comprises a tongue-and-groove connection.

3. The prosthetic orthopedic implant assembly according to claim 2, wherein the tongue-and-groove connection comprises at least one indentation on an exterior surface of the implant body and at least one protrusion on an interior surface of the elastomeric cover and wherein the at least one protrusion is received in the at least one indentation.

4. The prosthetic orthopedic implant assembly according to claim 3, wherein the at least one indentation comprises an annular groove on the exterior surface of the implant body.

5. The prosthetic orthopedic implant assembly according to claim 1, wherein the implant body is ball-shaped, wherein the elastomeric cover covers at least a portion of the ball-shaped implant body, and wherein the ball-shaped implant body includes a ridge encircling a portion of the implant body adapted to prevent said elastomeric cover from becoming detached from an exterior surface of said implant body.

6. The prosthetic orthopedic implant assembly according to claim 1, wherein the implant body is ball-shaped and includes a tooth for contacting an inner surface of the elastomeric cover, wherein the elastomeric cover covers at least a portion of said ball-shaped implant body, and wherein the contact between the tooth and the inner surface of the elastomeric body forms a fluid-tight seal for preventing fluid from seeping between the elastomeric cover and the implant body.

7. The prosthetic orthopedic implant assembly according to claim 6, wherein the mechanical fastener comprises a tongue-and-groove connection between the implant body and the elastomeric cover, and wherein the tooth protrudes from a groove portion of the tongue-and-groove connection.

8. The prosthetic orthopedic implant assembly according to claim 1, wherein the mechanical fastener comprises a retaining ring for retaining the elastomeric cover in position on the implant body.

9. The prosthetic orthopedic implant assembly according to claim 8, wherein the retaining ring is secured to a non-articulating surface of the implant body with a mechanical fastener.

10. The prosthetic orthopedic implant assembly according to claim 8, wherein the retaining ring is formed with an at least partial annular ridge that mates with an at least partial annular groove formed in at least one of the elastomer cover and the implant body.

11. The prosthetic orthopedic implant assembly according to claim 8, wherein the retaining ring permits local relative motion between the elastomeric cover and the implant body.

12. The prosthetic orthopedic implant assembly according to claim 1, wherein the implant body comprises a plate portion having at least one mounting member that protrudes from an interior surface thereof, and at least one fastener on an exterior surface thereof for fastening the elastomeric cover to the implant body.

13. The prosthetic orthopedic implant assembly according to claim 12, wherein the at least one fastener includes an at least partial annular lip spaced from the plate portion by an at least partially annular spacer ring, and the elastomeric cover comprises an inwardly facing lip that fits into a gap between the at least partially annular lip and the plate portion.

14. The prosthetic orthopedic implant assembly according to claim 13, wherein the at least partially annular lip, the plate portion and the at least partial spacer ring are formed of one integral body.

15. The prosthetic orthopedic implant assembly according to claim 13, wherein the at least partially annular lip, the plate portion and the at least partial spacer ring are formed of separate pieces, and the elastomeric cover comprises a partition wall that fits into a gap between separate pieces.

16. The prosthetic orthopedic implant assembly according to claim 12, further comprising an inner core positioned inwards of the elastomeric cover.

17. The prosthetic orthopedic implant assembly according to claim 16, wherein the inner core is constructed of a material with different properties than the elastomeric cover.

18. The prosthetic orthopedic implant assembly according to claim 17, wherein the inner core is made of a material that is more compliant and more resilient than the material of the elastomeric cover.

19. The prosthetic orthopedic implant assembly according to claim 1, wherein the mechanical fastener includes a threaded fastener that fastens the elastomeric cover to the implant body.

20. The prosthetic orthopedic implant assembly according to claim 19, wherein the threaded fastener comprises a threaded ring that mates with complementary formed threads on the implant body.
